(19) United States
(12) Patent Application Publication
(10) Pub. No.: US 2025/0340807 A1
(43) Pub. Date: Nov. 6, 2025

(54) BIOPROCESSING APPARATUS AND SYSTEM HAVING A PROBE SUPPORT PLATE

(71) Applicant: Global Life Sciences Solutions USA LLC, Marlborough, MA (US)

(72) Inventor: Yann Gandon, Portsmouth (GB)

(73) Assignee: Global Life Sciences Solutions USA LLC, Marlborough, MA (US)

(21) Appl. No.: 18/654,544

(22) Filed: May 3, 2024

(51) Int. Cl. C12M 1/12; C12M 1/00

(52) U.S. Cl. CPC C12M 23/06; C12M 23/26; C12M 23/28

(57) ABSTRACT

A rigid support structure for supporting a flexible bioprocessing vessel includes a body portion having an interior that includes a bottom surface and a substantially open top, interior configured to receive a flexible bioprocessing vessel. The structure further includes an opening in a front surface of the body portion allowing access to the interior, the opening being in proximity to the bottom surface of the interior. The structure further includes a probe support plate located at a lower portion of the opening. Wherein the probe support plate includes a plurality of apertures configured to receive and support a plurality of probes operatively connected to a flexible bioprocessing vessel within the interior.

### BACKGROUND

### Technical Field

Embodiments of the invention relate generally to bioprocessing, and, more particularly, to an apparatus, system and method of bioprocessing utilizing a rigid support structure having a probe support plate.

### Discussion of Art

Mixers and bioreactors are often employed to carry out biochemical and biological processes and/or manipulate liquids and other products of such processes. These devices typically utilize single-use vessels e.g., flexible or collapsible bags that are supported by an outer rigid structure such as a stainless-steel housing/tank.

In use, a disposable/single-use bag is positioned within the rigid tank and filled with the desired fluid for processing. An impeller assembly that includes a rotating impeller having one or more blades is disposed within the bag and is used to mix the fluid. Existing impeller systems are either top-driven, having a shaft that extends downwardly into the bag, on which one or more impellers are mounted, or bottom-driven, having an impeller disposed in the bottom of the bag that is driven by, for example, a magnetic drive system positioned outside the bag.

As will be appreciated, processes carried out within such bag generally involve the addition and/or removal of fluids before, during and/or after such processes. This necessitates the inclusion of multiple ports on the bag. Moreover, probes that measure various process parameters are often utilized which also necessitate ports and/or fittings. For example, such bags may include multiple fluid inputs/connectors/ports for exhaust filtering, sensing, and adding fluids to the interior cavity of the bag. In certain bags, the fluid inputs/connectors/ports are located in one or more rows on the front of the vessel as well as on a top surface thereof.

As mentioned, these bags are positioned within rigid support structures, e.g., stainless steel tanks, during use. The tanks may be relatively large, having capacities of 2000 L-3000 L or more. Many current tanks are cylindrical structures that include a door that is selectively openable. During use, a user places a bag within the open tank, connects the various ports to fluid sources, sensors, and the like, and deploys the bag within the now closed tank for use. Known tanks may include a cut away portion in the door so that, when the door is closed, tubing or sensors connected to ports or fittings on the front of the bag can extend out of the tank and/or otherwise be accessed while the tank door is closed.

As stated, the bags configured for use within such tanks are flexible and quite large, e.g., 2000 L or more. These bags have a flexible front surface or panel on which the fluid and sensor inputs/connectors/ports are located. While known tanks have cut away portions in the doors (or in a tank wall if no doors are present), these tanks lack structural elements that support the ports and fittings located on the front panel. As such, when deployed and during use, probes and the like are unsupported on the front surface of the bag. Moreover, the bag may be prone to bulge out of the cutaway portion when the door is closed.

Moreover, known tanks do not include features that facilitate alignment and/or prevent rotation of a flexible vessel within a rigid support structure/tank during installation and deployment. Given the relatively unwieldy size of some flexible vessels, alignment may be challenging.

In view of the above, there is a need for a bioprocessing apparatus and system that supports probes and the like that are connected to or located on a surface of a flexible bag during use. There is also a need for a bioprocessing apparatus and system that reduces bulging of a flexible bag during use.

### BRIEF DESCRIPTION

Certain embodiments commensurate in scope with the originally claimed subject matter are summarized below. These embodiments are not intended to limit the scope of the claimed subject matter, but rather these embodiments are intended only to provide a brief summary of the possible embodiments. Indeed, the disclosure may encompass a variety of forms that may be similar to or different from the embodiments set forth below.

In an aspect, a rigid support structure for supporting a flexible bioprocessing vessel includes a body portion having an interior that includes a bottom surface and a substantially open top, the interior configured to receive a flexible bioprocessing vessel. The structure further includes an opening in a front surface of the body portion allowing access to the interior, the opening being in proximity to the bottom surface of the interior and a probe support plate located at a lower portion of the opening. Wherein the probe support plate includes a plurality of apertures configured to receive and support a plurality of probes operatively connected to a flexible bioprocessing vessel within the interior.

In an embodiment, the plurality of apertures are a series of teeth having substantially U-shaped probe support surfaces between the teeth.

In an embodiment, the body portion further includes a door that may be selectively opened or closed to allow access to the interior through the opening.

In an embodiment, the door includes a brace portion that, when the door is closed, contacts a portion of the plurality of apertures to brace them.

In an embodiment, the brace portion includes an opening allowing access to additional ports, probes, and/or tubing of the flexible support vessel located above the probes supported by the probe support plate.

In an embodiment, the probe support plate includes a central cut out configured to receive a tab of an apparatus for transporting and/or installing a flexible bioprocessing vessel.

In an embodiment, the probe support plate is manufactured from a stainless steel welded to the interior of the body portion.

In an embodiment, the interior of the body portion is cylindrical.

In an embodiment, the probe support plate has a radius of curvature that substantially matches a radius of curvature of the cylindrical interior of the body portion.

In another aspect, a system for bioprocessing includes a flexible bioprocessing vessel that has a base portion that is attached to a bottom surface of the flexible bioprocessing vessel, the base portion including a front panel that includes a plurality of apertures configured to secure and protect a plurality of probes extending from the flexible bioprocessing vessel. The system further includes a rigid support structure having a body portion having an interior that includes a bottom surface and a substantially open top. The structure includes an opening in a front surface of the body portion allowing access to the interior, the opening being in proximity to the bottom surface of the interior and a probe support plate located at a lower portion of the opening, the probe support plate having a plurality of apertures. Wherein when the flexible bioprocessing vessel is installed in the interior of the body portion, the probe support plate contacts the front panel of the base portion, and the plurality of apertures of the probe support plate and plurality of apertures of the front panel are substantially aligned providing additional support to the plurality of probes operatively connected to the flexible bioprocessing vessel.

In an embodiment, the plurality of apertures of the probe support plate are a series of teeth having substantially U-shaped probe support surfaces between the teeth.

In an embodiment, the body portion further includes a door that may be selectively opened or closed to allow access to the interior through the opening.

In an embodiment, the door includes a brace portion that, when the door is closed, contacts a portion of the plurality of apertures of the probe support plate to brace them during use.

In an embodiment, the brace portion includes an opening allowing access to additional ports, probes, sensors, and/or tubing of the flexible support vessel located above the plurality of probes supported by the front panel and the probe support plate.

In an embodiment, the probe support plate includes a central cut out configured to receive a tab of an apparatus for transporting and/or installing a flexible bioprocessing vessel.

In an embodiment, the interior of the body portion is cylindrical.

In another aspect, a method for installing a flexible bioprocessing vessel within a rigid support structure includes placing the flexible bioprocessing vessel into an interior of the rigid support structure, the rigid support structure having a probe support plate and placing a probe, sensor, or fluid line of the flexible bioprocessing vessel into an aperture of a probe support plate. The method further includes deploying the flexible bioprocessing vessel so that it may process fluid within an interior of flexible bioprocessing vessel.

In an embodiment, the method further includes closing a door of the rigid support structure, the door including a brace portion that, when closed, contacts a portion of the aperture of the probe support plate to brace it during use.

The rigid support structure of claim 17 wherein the aperture is a U-shaped probe support surfaces located between two teeth.

### BRIEF DESCRIPTION OF THE FIGURES

The present invention will be better understood from reading the following description of non-limiting embodiments, with reference to the attached drawings, wherein below:

FIG. 1 is a perspective view of a rigid support structure and flexible vessel;

FIG. 2 is a perspective view of a flexible vessel suitable for use with embodiments of the invention;

FIG. 3 is a top perspective view of an apparatus for transporting and installing a flexible bioprocessing vessel suitable for use with embodiments of the invention;

FIG. 4 is an enlarged perspective illustration of a probe support plate of a rigid support structure in accordance with an embodiment of the invention;

FIG. 5 an enlarged perspective illustration of the rigid support structure of FIG. 2 with a door of the support structure closed illustrating a brace portion in accordance with an embodiment of the invention;

FIG. 6 is an enlarged perspective view of the support structure of FIG. 4 in a closed door configuration illustrating a vessel, probes, probe support plate and brace portion according to an embodiment of the invention; and

FIG. 7 an enlarged perspective view of the rigid support structure of FIG. 4 in an open-door configuration illustrating a vessel, probes, and probe support plate according to an embodiment of the invention.

### DETAILED DESCRIPTION

Reference will be made below in detail to exemplary embodiments of the invention, examples of which are illustrated in the accompanying drawings. Wherever possible, the same reference characters used throughout the drawings refer to the same or like parts.

As used herein, the term “flexible” or “collapsible” refers to a structure or material that is pliable, or capable of being bent without breaking, and may also refer to a material that is compressible or expandable. An example of a flexible structure is a bag formed of polyethylene film.

A “vessel,” as the term is used herein, means a flexible bag, a flexible container, a semi-rigid container, or a rigid container, as the case may be. The term “vessel” as used herein is intended to encompass bioprocessing vessels having a wall or a portion of a wall that is flexible, single-use flexible bags, as well as other containers or conduits commonly used in biological or chemical processing, including, for example, cell culture/purification systems, fermentation systems, mixing systems, media/buffer preparation systems, and filtration/purification systems.

As used herein, the term “bag” means a flexible or semi-rigid vessel used, for example, as a mixer or bioreactor for the contents within.

Embodiments may be utilized in connection with a wide variety of biological and chemical processes, which are referred to generally herein as “bioprocessing.” This term encompasses, but is not limited to, the various processes that occur in bioreactors, mixers, fermenters, and the like. A “bioprocessing vessel” is a vessel suitable for use with or in a bioreactor, mixer, fermenter, or other biological or chemical processing device.

While embodiments may be particularly suitable for use with relatively large rigid support structures and flexible vessels, e.g., 2000 L, 3000 L or larger, the invention is not limited in this regard. Embodiments may also be suitable for use with smaller structures/vessels, e.g., 500 L.

Although embodiments are described as for use with cylindrical reactor tanks, embodiments may be suitable for use with tanks (and vessels that fit therein) having other geometries, e.g., square, rectangular, and hexagonal.

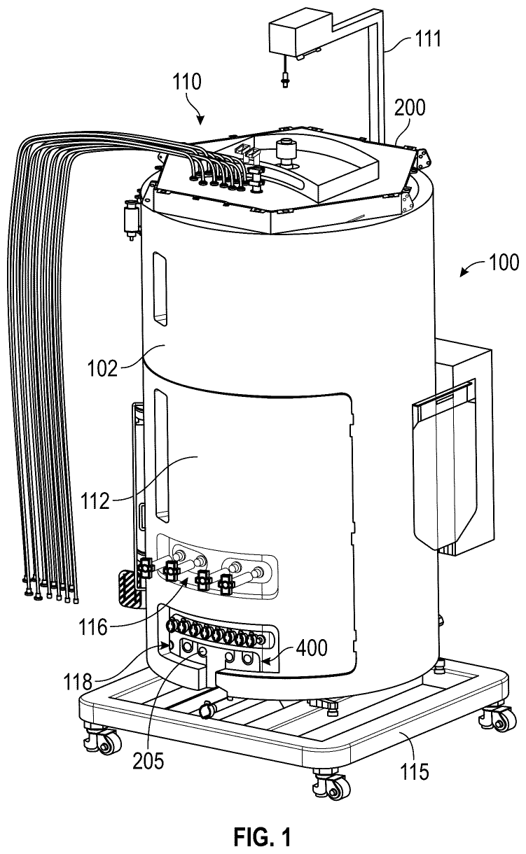

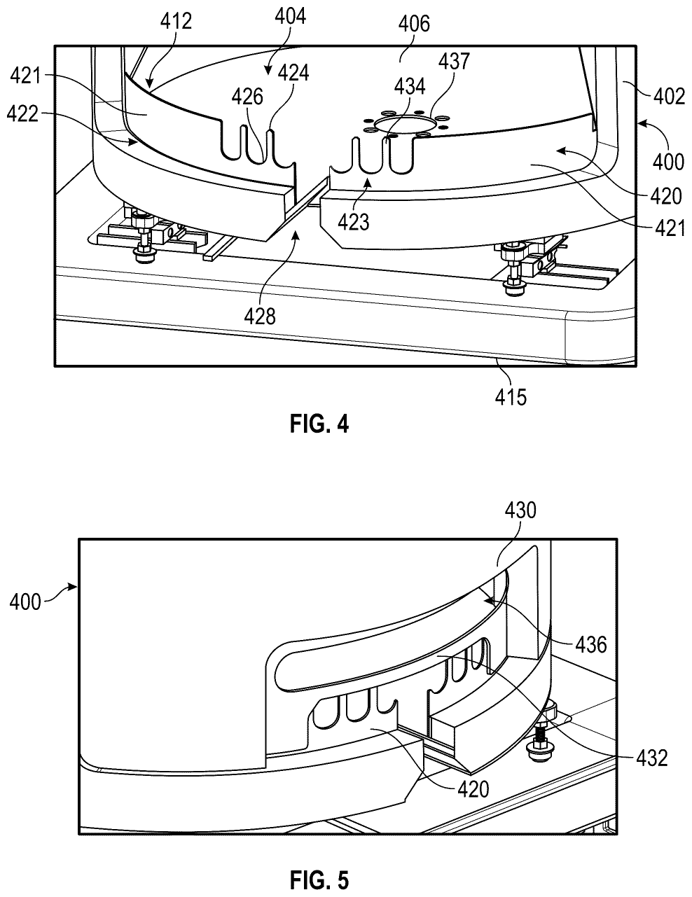

Referring now to FIG. 1 and FIG. 4, an exemplary tank/rigid support structure 100 is depicted. The rigid support structure 100 includes a rigid body 102 having an interior 404 that includes a bottom panel 406. The interior 404, which is cylindrical, has a substantially open top 110 and a selectively openable door 112 which allows access to the interior 104, which is open and configured to receive a flexible bioprocessing vessel 200. The rigid support structure 100 further includes a stand portion 115 attached to the rigid body 102 which allows for access to the space below the rigid body 102.

In embodiments, the rigid support structure 100 may further include a hoist mechanism 111 to mechanically lift a vessel to assist in the installation of the vessel within the structure. As will be appreciated, such a mechanism may be particularly suitable for use with larger vessels and support structure, e.g., 2000 L vessels and larger.

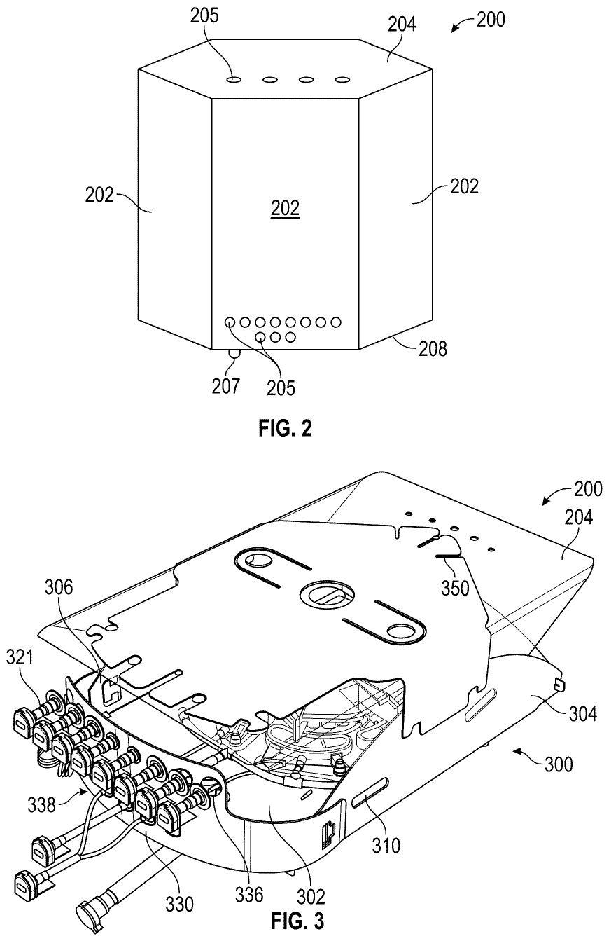

As shown in FIG. 2, suitable vessels for use with embodiments of the invention include a flexible bioprocessing vessel 200. In the depicted embodiment, the flexible bioprocessing vessel 200 includes at least eight flexible panels forming sides, top and bottom of the flexible bioprocessing vessel, e.g., six flexible side panels 202, a top panel 204, and a bottom panel 208 located on an opposite end of the flexible bioprocessing vessel 200 from the top panel 204. The eight flexible panels are joined together, e.g., welded, to define an interior configured for processing a fluid.

Although embodiments of the invention are shown for use with hexagonal vessels located within cylindrical support structures, embodiments are not limited to specific bag geometries.

The flexible bioprocessing vessel 200 includes a plurality of fluid inputs/connectors/ports 205 for exhaust filtering, sensing, and adding fluids to the interior cavity, by way of example. In embodiments, the fluid inputs/connectors/ports 205 are located in one or more rows on a front facing flexible panel of the vessel 200, and on the top panel 204. As will be appreciated, the vessel 200 includes at least one fluid output 207 for fluid removal.

Turning now to FIG. 3, flexible bioprocessing vessel 200 may be used with an apparatus 300 for transporting the vessel and installing the vessel within the rigid support structure 100 (FIG. 1). The apparatus 300 includes a base portion 302 which is configured for operative connection to a bottom panel 208 of the vessel 200. The base portion 302 includes one or more handles 310 on each side of the base portion 302 facilitating lifting the vessel 200 from external packaging and placing/installing the vessel 200 within a rigid support structure 100 for deployment.

As depicted, the apparatus 300 also includes a front panel 330 attached to the base portion 302. In embodiments, the front panel 330 includes a plurality of apertures 332 (see, e.g., FIG. 7) configured to secure and protect tubing, connectors, and the like, extending from the flexible bioprocessing vessel 200 during transportation and/or use. In embodiments, there are two rows of apertures 332 that extend across the front panel 330; an upper row 336 and a lower row 338 which correspond to the location and number of tubing lines and/or connectors 321 on the flexible bioprocessing vessel 200 that may extend from the vessel. As shown, the front panel 330 may have a curved profile that substantially matches a radius of curvature of an interior surface of the rigid support structure 102, facilitating installation of the vessel 200 within the rigid support structure 102. The curved profile of the front panel 330 may also match a radius of curvature of the front end 360 of the base portion 302.

Referring back to FIG. 1, the door 112 of the support structure 100 includes an upper cut away portion 116 and a lower cut away portion 118. As will be appreciated, the cut away portions allow access to the inputs, connectors, and/or ports 205 located on the front facing surface of the flexible bioprocessing vessel 200 when the door is closed, e.g., during use. The cut away portions, however, may allow the vessel to bulge/protrude outward when it has fluid within its interior cavity. Moreover, the lower cut away portion 118 does not provide structural support for probes or fluid lines connected to ports 205 located on the vessel 200.

When a vessel 200 is used with apparatus 300, the front panel 330 provides fluid line/probe support. However, the apparatus 300 is also flexible and additional support to reduce/prevent vessel bulging at the cut away portions of the rigid support structure 100 is desirable.

Referring now to FIG. 4 and FIG. 5, a portion of a rigid support structure 400 for supporting a flexible bioprocessing vessel according to an embodiment is depicted. Much like the structure 100 of FIG. 1, the structure 400 includes a body portion 402 connected to a stand portion 115, the body portion 402 having an interior 404 that includes a bottom surface 406 and a substantially open top, the interior 404, which in the depicted embodiment is cylindrical, is configured to receive a flexible bioprocessing vessel 200.

The body portion 402 also includes an opening 412 in a front surface of the body portion which allows access to the interior 404. The opening 412 being in proximity to the bottom surface 406 of the interior 404. As shown, and in accordance with an embodiment, the body portion 402 includes a probe support plate 420 located at a lower portion 422 of the opening 412. The probe support plate 420 may extend across substantially an entirety of the lower portion 422 of the opening 412 from one side of to another, though there may be discontinuities/interruptions in the plate 420 such as the central opening/channel/cut out 428 discussed below.

The probe support plate includes a flange portion or lip 421. The lip 421 contacts the flexible vessel 200 during use and helps to prevent bulging of the vessel 200 out of the opening 412. In embodiments, the height of the lip 421 may vary.

In the depicted embodiment, the probe support plate 420 is manufactured from the same rigid material as the support structure 400, e.g., stainless steel. The probe support plate 420 may be welded to the lower portion 422 of the opening 412 or may be a unitary with the body portion 402. In other embodiments, the probe support plate 420 may be removably fastened to the body portion 402. Indeed, it may be possible to have several probe support plates 420 for use with vessels having different configurations, numbers, and/or orientations of ports. When a different vessel type is used, a different support plate may be secured to the body portion 402. While the body portion 402 and probe support plate 420 are disclosed as metal, in other embodiments they may be manufactured from a rigid polymeric material.

The probe support plate 420 includes a plurality of apertures 423 configured to receive and support a plurality of probes operatively connected to a vessel 200 within the interior 404. As depicted, the plurality of apertures 423 are formed via a series of teeth 424 having substantially U-shaped probe support surfaces 426 between the teeth 424. The apertures 423 have open tops such that probes or other vessel connections/fixtures may be dropped/lowered into the aperture 423 and into contact with probe support surfaces 426.

The size, number, and placement of the apertures 423 may vary depending on the vessel and/or probes and the like that are to be used. In the depicted embodiment, the probe support plate 420 includes six probe support surfaces 426 formed by four teeth 424. The probe support plate 420 also includes a channel or central cut out 428 that extends into the interior 404 of the rigid support structure 400. In particular, the channel/cut out 428 extends along the bottom surface 406 of the interior 404 of the rigid support structure 400. The cut out 428 is open to the exterior space below the rigid support structure 400.

Referring now to FIGS. 4-7, the central cut out 428 is configured to receive a tab 340 located on the front panel 330 of apparatus 300. When installing a flexible bioprocessing vessel 200 that is operatively connected to the apparatus 300, the tab 340 is placed within the channel/central cut out 428 and the apparatus 300 and vessel 200 are moved into the interior of the rigid support structure 400 through the open door. In embodiments, the central cut out 428 is substantially U-shaped, C-shaped, or rectangular in profile, with a terminal end that the tab 340 may contact when fully installed, in certain embodiments. As will be appreciated, the tab 340 and cut out 428 prevent the apparatus 300 and an attached vessel 200 from rotating in the rigid support structure 400. In embodiments, the cut-out 428 may connect to the aperture 437 for the impeller motor (FIG. 4). That is, the cut-out may form a key-hole shaped structure that terminates in the aperture 437 instead of a terminal end located in front of the aperture 437, i.e. closer to the door 112.

As depicted, in an embodiment, the rigid support structure 400 and the interior 404 of the body portion 402 of the rigid support structure 400 are cylindrical. In such embodiments, the probe support plate 420 may have a radius of curvature that substantially matches a radius of curvature of the interior 404 of the body portion 402.

As will be appreciated, however, the interior of the body portion need not be cylindrical and embodiments may be used with rigid support structures that are, for example, square, rectangular, hexagonal, and other geometries. In these embodiments, the probe support plate may be flat as opposed to curved.

Turning now to FIGS. 4-6, the body portion 402 further includes a door 430 that may be selectively opened or closed to allow access to the interior 404 through the opening 412. In embodiments, the door 430 has a locking mechanism and may include one or more windows (not shown). The door 430 includes a brace portion 432 that, when the door 430 is closed, contacts an upper portion 434 of the plurality of apertures (e.g., an end portion of the teeth) to brace them. The brace portion 432 may be welded to the door 430. In other embodiments, the brace portion 432 may be fastened to the door 430 and may be selective removable. In other embodiments, the brace portion 432 may be omitted.

As depicted, the brace portion 432 includes an opening 436 allowing access to additional ports, probes, and/or tubing of the flexible support vessel located above the probes supported by the probe support plate. In certain embodiments, the opening 436 may include probe supports (not shown). Such probe supports may be formed via spaced apart teeth as with the probe support plate 420.

The opening 436 may be sized and shaped based on vessel probe location and number. In certain embodiments there may be no opening 436 in the brace portion 432. In embodiments, the probe support plate 420 and the brace portion 432 may be located on the door 430.

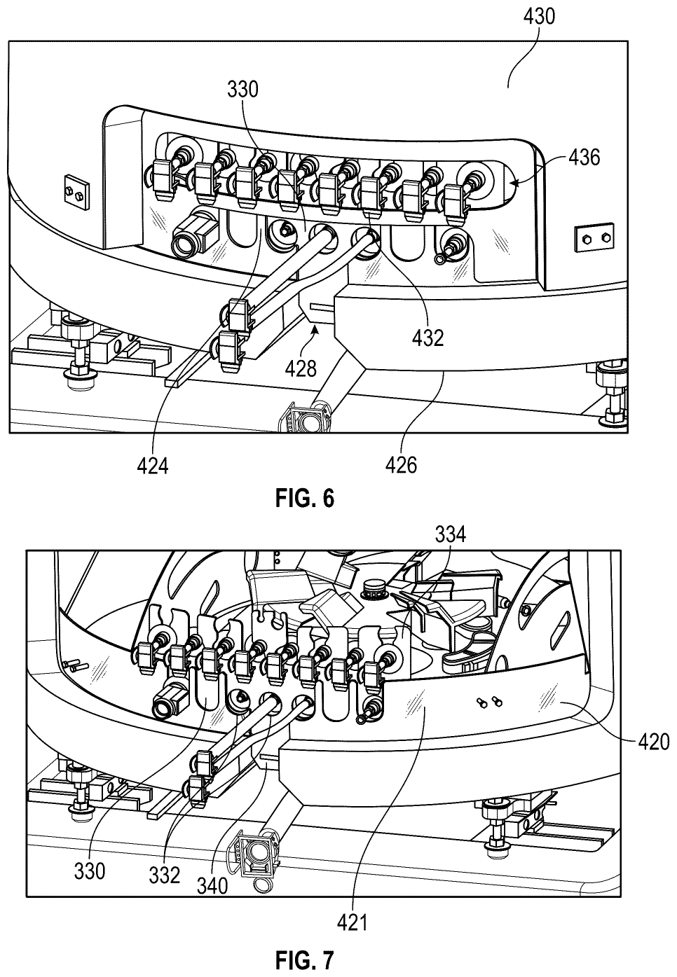

Referring to FIG. 6 and FIG. 7, as mentioned, embodiments may be used with flexible vessels 200 that are operatively connected to an apparatus 300 for transporting and installing the vessel 200 within a rigid support structure. In particular, the front panel 330 of the apparatus 300 has apertures 332 and slots 334 that receive ports on the front of the vessel. The apertures 332 and slots 334 support probes and tubing.

As shown, in embodiments, the apertures 332 and slots 334 are substantially aligned with the probe support plate 420 and opening 436. Thus, the probe support plate 420 may provide support to probes that extend through the apertures 332 of the apparatus 300. The brace portion 432 may contact/support probes, lines or connectors that extend through the slots 334 and out of the opening 436.

Embodiments of the invention contemplate a method for installing a flexible bioprocessing vessel 200 within a rigid support structure. The method involves placing the flexible bioprocessing vessel 200 into an interior 404 of the rigid support structure, the rigid support structure having a probe support plate 420 and placing a probe, sensor, or fluid line of the flexible bioprocessing vessel into an aperture of a probe support plate 420. The method further includes deploying the flexible bioprocessing vessel so that it may process fluid within an interior of flexible bioprocessing vessel.

The method further includes closing a door of the rigid support structure, the door including a brace portion that, when closed, contacts a portion of the plurality of apertures of the probe support plate to brace them during use.

The aperture is a U-shaped probe support surfaces located between two teeth.

As used herein, an element or step recited in the singular and proceeded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional such elements not having that property.

While the dimensions and types of materials described herein are intended to define the parameters of the invention, they are by no means limiting and are exemplary embodiments. Many other embodiments will be apparent to those of skill in the art upon reviewing the above description.

The scope of the invention should, therefore, be determined with reference to the appended claims, along with the full scope of equivalents to which such claims are entitled. In the appended claims, the terms “including” and “in which” are used as the plain-English equivalents of the respective terms “comprising” and “wherein.”

Moreover, in the following claims, terms such as “first,” “second,” “upper,” “lower,” “bottom,” “top,” etc. are used merely as labels, and are not intended to impose numerical or positional requirements on their objects. Further, the limitations of the following claims are not written in means-plus-function format and are not intended to be interpreted as such, unless and until such claim limitations expressly use the phrase “means for” followed by a statement of function void of further structure.

This written description uses examples to disclose several embodiments of the invention, including the best mode, and also to enable one of ordinary skill in the art to practice the embodiments of invention, including making and using any devices or systems and performing any incorporated methods. The patentable scope of the invention is defined by the claims, and may include other examples that occur to one of ordinary skill in the art. Such other examples are intended to be within the scope of the claims if they have structural elements that do not differ from the literal language of the claims, or if they include equivalent structural elements with insubstantial differences from the literal language of the claims.

1. A rigid support structure for supporting a flexible bioprocessing vessel comprising:
a body portion having an interior that includes a bottom surface and a substantially open top, the interior configured to receive a flexible bioprocessing vessel;
an opening in a front surface of the body portion allowing access to the interior, the opening being in proximity to the bottom surface of the interior; and
a probe support plate located at a lower portion of the opening;
wherein the probe support plate includes a plurality of apertures configured to receive and support a plurality of probes operatively connected to a flexible bioprocessing vessel within the interior.

2. The rigid support structure of claim 1, wherein the plurality of apertures are a series of teeth having substantially U-shaped probe support surfaces between the teeth.

3. The rigid support structure of claim 1, wherein the body portion further includes a door that may be selectively opened or closed to allow access to the interior through the opening.

4. The rigid support structure of claim 3, wherein the door includes a brace portion that, when the door is closed, contacts a portion of the plurality of apertures to brace them.

5. The rigid support structure of claim 4, wherein the brace portion includes an opening allowing access to additional ports, probes, and/or tubing of a flexible bioprocessing vessel located above the probes supported by the probe support plate.

6. The rigid support structure of claim 1, wherein the probe support plate includes a central cut out configured to receive a tab of an apparatus for transporting and/or installing the flexible bioprocessing vessel.

7. The rigid support structure of claim 1, wherein the probe support plate is manufactured from a stainless steel welded to the interior of the body portion.

8. The rigid support structure of claim 1, wherein the interior of the body portion is cylindrical.

9. The rigid support structure of claim 8, wherein the probe support plate has a radius of curvature that substantially matches a radius of curvature of the interior of the body portion.

10. A system for bioprocessing comprising:
a flexible bioprocessing vessel that includes a base portion that is attached to a bottom surface of the flexible bioprocessing vessel, the base portion including a front panel that includes a plurality of apertures configured to secure and/or protect a plurality of probes extending from the flexible bioprocessing vessel; and
a rigid support structure comprising:
a body portion having an interior that includes a bottom surface and a substantially open top;
an opening in a front surface of the body portion allowing access to the interior, the opening being in proximity to the bottom surface of the interior; and
a probe support plate located at a lower portion of the opening, the probe support plate having a plurality of apertures;
wherein when the flexible bioprocessing vessel is installed in the interior of the body portion, the probe support plate contacts the front panel of the base portion, and the plurality of apertures of the probe support plate and plurality of apertures of the front panel are substantially aligned providing additional support to the plurality of probes operatively connected to the flexible bioprocessing vessel.

11. The system of claim 10 wherein the plurality of apertures of the probe support plate are a series of teeth having substantially U-shaped probe support surfaces between the teeth.

12. The system of claim 10 wherein the body portion further includes a door that may be selectively opened or closed to allow access to the interior through the opening.

13. The system of claim 12, wherein the door includes a brace portion that, when the door is closed, contacts a portion of the plurality of apertures of the probe support plate to brace them during use.

14. The system of claim 13, wherein the brace portion includes an opening allowing access to additional ports, probes, sensors, and/or tubing of the flexible bioprocessing vessel located above the plurality of probes supported by the front panel and the probe support plate.

15. The system of claim 10 wherein the probe support plate includes a central cut out configured to receive a tab of an apparatus for transporting and/or installing a flexible bioprocessing vessel.

16. The system of claim 10 wherein the interior of the body portion is cylindrical.

17. A method for installing a flexible bioprocessing vessel within a rigid support structure comprising:
placing the flexible bioprocessing vessel into an interior of the rigid support structure, the rigid support structure having a probe support plate;
placing a probe, sensor, or fluid line of the flexible bioprocessing vessel into an aperture of a probe support plate; and
deploying the flexible bioprocessing vessel so that it may process fluid within an interior of flexible bioprocessing vessel.

18. The method of claim 17 further comprising:
closing a door of the rigid support structure. the door including a brace portion that, when closed, contacts a portion of the aperture of the probe support plate to brace it during use.

19. The method of claim 17 wherein the aperture is a U-shaped probe support surfaces located between two teeth.
